(19) United States
(12) Patent Application Publication
(10) Pub. No.: US 2002/0022024 A1
(43) Pub. Date: Feb. 21, 2002

(54) Hemoglobinase inhibitors and methods of use

(76) Inventors: Salman Baig, Athens, GA (US); David S. Peterson, Athens, GA (US)

Correspondence Address: MUETING, RAASCH & GEBHARDT, P.A., P.O. BOX 581415, MINNEAPOLIS, MN, 55458, US

(21) Appl. No.: 09/826,177

(22) Filed: Apr. 4, 2001

Related U.S. Application Data
(60) Provisional application No. 60/194,426, filed on Apr. 4, 2000.

(51) Int. Cl. A61K 38/48

(52) U.S. Cl. 424/94.63

(57) ABSTRACT

The present invention provides methods for the identification of inhibitors of hemoglobinases produced by parasites and methods for the use of such inhibitors. Also provided are methods of treating animals that are at risk of a parasitic infection, and methods of immunizing an animal at risk of a parasitic infection.

### CONTINUING APPLICATION DATA

This application claims the benefit of U.S. Provisional Application Ser. No. 60/194,426, filed Apr. 4, 2000, which is incorporated by reference herein.

### BACKGROUND

Helminths, or parasitic worms, infect and cause a wide variety of diseases in humans and livestock worldwide. The economic loss caused by helminths is great. In the United States alone, the estimated annual losses in cattle, sheep, goat, swine, horse, and poultry production due to internal helminth infections is $1.2 trillion. The estimated annual expenditures for chemotherapeutic agents to prevent helminth infections in poultry, horses, ruminants, and swine is $475 million. The annual economic cost to humans in the United States, in terms of public health expenditures and lost wages, has been estimated to be at least $216 million. Worldwide, the number of humans infected with helminths is substantial, as are the economic costs and adverse effects on human morbidity and mortality. It is estimated that worldwide about 1.55 million people are infected with Schistosoma japonicum, and about 83 million people are infected with Schistosoma mansoni. Schistosomiasis kills about 250,000 people annually.

Macrocyclic lactones are the drugs most commonly used to prevent helminth infection. Examples of macrocyclic lactone antihelmintics include avermectins and milbemycins. Other classes of antihelmintic drugs include the benzimidazoles, and the acetylcholine receptor agonists levamisole, pyrantel, and morantel.

According to Geary et al., (Vet. Parasitol., 84, 275-295 (1999)), there appear to be only about three new classes of drugs that may be useful to treat livestock, the diketopiperazines, cyclic depsipeptides, and nitozoxanide. However, it is believed that no compounds from any of these classes have reached clinical development.

There is evidence that helminths develop resistance to many of the veterinary drugs, including the avermectins and milbemycins. The development of resistance can lead to failure of preventative treatment and the resulting establishment of the resistant parasite in an animal. Genetically resistant strains pass on their alleles to subsequent generations, and this pattern is inherited. Resistance was first noticed in sheep and horse parasites, but it has now appeared in many animal populations, including pigs, sheep and humans. For example, resistance to the hycanthone/oxamiquine class of drugs has been detected in human schistosomiasis.

Of greatest concern is development of resistance to the avermectins and milbemycins. In vivo studies have demonstrated that parasites resistant to avermectins are also commonly resistant to the related milbemycins, suggesting a common mechanism of action (Sangster et al., Parasitology Today, 15, 141-146 (1999)). It has been recognized that resistance of helminths to currently available drugs could become a major problem in the human population.

Helminths exhibit a wide range of feeding behaviors. Bloodfeeding or hemotophagous helminths use hemoglobin from the red blood cells of a host animal as a significant source of amino acids for nutrition. Bloodfeeding hehninths use enzymes called “hemoglobinases” to degrade hemoglobin. The most studied hemoglobinases are from the plasmodia parasites, protozoans which causes malaria. In this pathway of hemoglobin breakdown by parasites, it is postulated that a series of enzymes are employed to degrade hemoglobin. It appears that this pathway is also rather conserved among the hemoglobinases of the helminth bloodfeeder, Schistosoma mansoni, whose hemoglobinases are the best studied of the helminths. The amount of hemoglobin used by bloodfeeding helminths can be substantial. For instance, a female schistosome ingests about 330,000 red blood cells per hour.

The first event in the hemoglobin degradation pathway is postulated to be the lysis of red blood cells in the helminth esophagus by a hemolysin to release hemoglobin. Hemoglobin is then thought to be transported down to the schistosome cecum, and subsequently cleaved by secretory endoprotenases (including cathepsin D, cathepsin Ls, and cathepsin B) into peptide fragments followed by its digestion into individual amino acids by secretory exo-protenases (including cathepsin C). Following this process, additional proteases are thought to play key roles in the remaining degradation. The secretory endoproteases involved in hemoglobin cleavage include a cathepsin B cysteine protease, which efficiently cleaves hemoglobin. Cathepsin B proteases are also found as lysosomal enzymes that play a fundamental role in normal cellular physiology. In contrast to parasite cathepsin B proteases that efficiently cleave hemoglobin, lysosomal cathepsin B proteases do not cleave hemoglobin as efficiently.

Cathepsin B proteases of S. mansoni were first noted for their immunodiagnostic potential (Ruppel et al., Clin. Exp. Immunol., 62, 499-506 (1985); Ruppel et al., Exp. Parasitol., 60, 195-206 (1985)), due to their successful reaction with antibodies from infected human and murine serum. Several in vivo studies have demonstrated the importance of cysteine proteases in the development of schistosomules, i.e., the form of the parasite immediately after invasion of a host and before the parasite becomes an adult. For example, it has been demonstrated that the addition of the cysteine protease inhibitor, EP459, to the culture media of schistosomules increased the death rate of schistosomules in comparison to controls (Zerda et al., Exp. Parasitol., 67, 238-246 (1988)). One of the major cysteine proteases involved in these degradations are the cathepsin Bs.

Using the coordinates of the crystal structure of human liver cathepsin B, a three dimensional model for the S. mansoni cathepsin B, Sm31, has been reported (Klinkert et al., FEBS Lett., 351, 397-400 (1994)). While the enzymes appear to be similar structurally, the models differ with respect to their inhibition by synthetic inhibitors. For example, when a short synthetic substrate is used, Z-Trp-Met-CHN2 appears to be a more potent inhibitor of Sm31 in comparison to a derivative of the irreversible cysteine protease inhibitor, E64.

In view of the worldwide adverse impact on human morbidity and mortality associated with helminth infections, as well as the economic costs and development of resistance by helminths, there is a continuing need for better ways to prevent infection by such parasites and treat animals that are infected with the parasites.

### SUMMARY OF THE INVENTION

The present invention represents an advance in the art of treating animals at risk of infection with parasites that rely on a host animal's hemoglobin as a nutrition source. Specifically, the invention facilitates the discovery of selective inhibitors of cathepsin B proteases found in bloodfeeding parasites.

During a comparison of the primary amino acid sequences of the active sites of cathepsin B cysteine proteases of helminths that were hypothesized to be hemoglobinases, a conserved motif was identified within the histidine active site and asparagine active site. Surprisingly and unexpectedly, the conserved motifs were found only in some cathepsin B proteases of parasites that use a host animal's hemoglobin as a source of nutrition, and not in other cathepsin B proteases. The presence of this motif in certain parasite cathepsin B proteases and its absence in cathepsin B proteases produced by a host animal makes possible the identification and use of inhibitors that inhibit parasite cathepsin B proteases but have little to no effect on host cathepsin B proteases.

Accordingly, the present invention provides methods for identifying an inhibitor of hemoglobinase activity, including incubating a solution containing a chemical entity, a hemoglobinase, and hemoglobin under conditions and for a time period suitable for the cleavage of the hemoglobin. The amount of hemoglobin remaining in the solution at the end of the time period is measured. The presence of more hemoglobin in the solution compared to a comparably treated solution that does not contain the chemical entity indicates the chemical entity is an inhibitor of hemoglobinase activity. The hemoglobinase can include an asparagine active site region. The asparagine active site region can include an amino acid sequence of SEQ ID NO:5.

The chemical entity can be a peptidomimetic, an organic compound, an inorganic compound, or a polypeptide, for instance a polyclonal antibody or a monoclonal antibody. The chemical entity can associate with at least one amino acid of an amino acid sequence depicted at SEQ ID NO:5 or at SEQ ID NO:6.

The methods can also include incubating a second solution that contains the inhibitor, a cathepsin B cysteine protease produced by a host animal (for instance human liver cathepsin B), and a substrate (for instance an a chain of hemoglobin) of the cathepsin B cysteine protease under conditions and for a time period suitable for the cleavage of the substrate. The amount of substrate remaining in the solution at the end of the time period is measured. Greater than 50% of the substrate added to the second solution is present after the incubation.

The present invention also provides methods for treating an animal, for instance a mammal, at risk of a parasite infection, for instance infection with a helminth, including administering to the animal an inhibitor that decreases the activity of a hemoglobinase. The hemoglobinase can include an asparagine active site region. The asparagine active site region can include an amino acid sequence of SEQ ID NO:5. The inhibitor can associate with at least one amino acid of an amino acid sequence of SEQ ID NO:5 or SEQ ID NO:6.

Another aspect of the invention provides methods of inhibiting the activity of a hemoglobinase including contacting the hemoglobinase with an inhibitor of hemoglobinase activity. The hemoglobinase can be in vitro or present in an animal.

The present invention also provides methods of immunizing an animal at risk of a parasitic infection, including a helminth infection. One method includes administering to the animal an antibody, for instance a polyclonal antibody or a monoclonal antibody, that associates with a region of a hemoglobinase. The region of the hemoglobinase can include at least one amino acid sequence depicted at SEQ ID NO:5 or SEQ ID NO:6. Another method includes administering to the animal a polypeptide that induces an immune response against a hemoglobinase expressed by a parasite. The polypeptide can include an amino acid sequence depicted at SEQ ID NO:5 or SEQ ID NO:6. The polypeptide can have an amino acid sequence selected from the group consisting of amino acid sequences of SEQ ID NO:5 and SEQ ID NO:6. The polypeptide can be present in a composition.

Also provided by the present invention are computer-assisted methods for identifying an inhibitor of parasite hemoglobinase activity, preferably a helminth hemoglobinase activity. The methods include supplying a computer modeling application with a set of structure coordinates of all or a portion of a parasite hemoglobinase. In some aspects, the methods further include supplying the computer modeling application with a set of structure coordinates of a chemical entity, and determining whether the chemical entity is an inhibitor expected to bind to or interfere with the hemoglobinase, wherein binding to or interfering with the hemoglobinase is indicative of potential inhibition of the hemoglobinase.

In other aspects, the computer-assisted methods further include supplying the computer modeling application with a set of structure coordinates of a chemical entity, structurally modifying the chemical entity to yield a set of structure coordinates for a modified chemical entity, and then determining whether the modified chemical entity is an inhibitor expected to bind to or interfere with the hemoglobinase, wherein binding to or interfering with the hemoglobinase is indicative of potential inhibition of the hemoglobinase.

In yet other aspects, the computer-assisted methods further include computationally building a chemical entity represented by a set of structure coordinates, and determining whether the chemical entity is an inhibitor expected to bind to or interfere with the hemoglobinase, wherein binding to or interfering with the hemoglobinase is indicative of potential inhibition of the hemoglobinase.

Unless otherwise specified, “a,” “an,” “the,” and “at least one” are used interchangeably and mean one or more than one.

### BRIEF DESCRIPTION OF THE FIGURES

FIG. 1. The histidine and asparagine active site signature regions for cysteine proteases, cathepsin B enzymes, and proteases with the hemoglobinase motif. a) Histidine active site region. Cysteine Proteases, the eleven amino acid consensus motif (SEQ ID NO:1) present in the histidine active site region of cysteine proteases; Motif, the eleven amino acid consensus motif (SEQ ID NO:6) present in the histidine active site region of bloodfeeder hemoglobinases; Position, the position of each amino acid in the consensus motifs. b) Asparagine active site region. Cysteine Proteases, the fourteen amino acid consensus motif (SEQ ID NO:7) present in the asparagine active site region of cysteine protease; Cathepsin B, the eight amino acid consensus motif (SEQ ID NO:2) present in cathepsin B proteases; Motif 1, the fourteen amino acid consensus motif (SEQ ID NO:5) present in helminth bloodfeeders; Motif 2, the fourteen amino acid consensus motif (SEQ ID NO:4) present in Aedes aegypti, Sarcophaga perigrina, Rattus norvegicus, human, and helminth bloodfeeders; Motif 3, the fourteen amino acid consensus motif (SEQ ID NO:3) present in protozoan bloodfeeders; Position, the position of each amino acid in the consensus motifs. Asterisks designate the catalytic residues. Brackets enclose the residues that can be found at one position. Within each pair of brackets, the residues that can be found at that position are separated by a “/”. X refers to an amino acid position that can be any amino acid. The cysteine protease motif pattern is displayed in PROSITE format (available at www.expasy.ch/ExpasyHunt/).

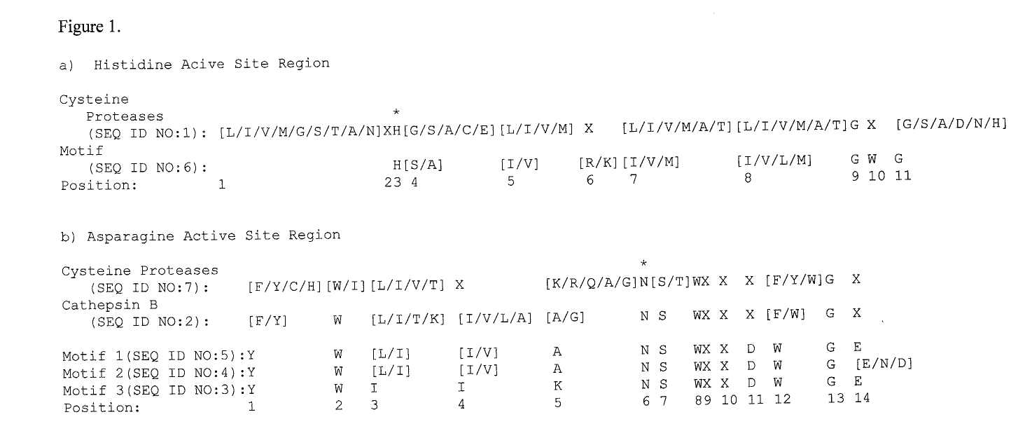

FIG. 2. Histidine and Asparagine active site regions of the papain family including cathepsin B and motif containing proteases. The conserved motif in the histidine active site region and the asparagine active site region of cathepsin B enzymes of bloodfeeders is boldfaced and boxed. At top, the P2 and P1′ substrate binding regions (i.e., of the substrate hemoglobinase molecule) are indicated, and the active site histidine and asparagine residues are indicated by an asterisk. Each dash represents a nonconserved residue. A. Cysteine proteases. B. Cathepsin B proteases. C. Helminth bloodfeeder cathepsin B hemoglobinases. Accession numbers from GenBank are in parentheses. Cysteine Proteases: Papain (CAB42883); Chymopapain (CAA66378); Caricain precursor (JN0633); Stem bromelain (S03964); Pea cysteine protease (P25804); Aleurain prec (P05167); human cathepsin H (NP_{—}004381); human cathepsin L (NP_{—}001903). Cathepsin B's: Mus musculus (CAA38713); R. norvegicus (CAA57792); T. aestevium (CAA46811); A. thaliana (AAC24376); C. elegans “gut specific cp” (P25807); C. elegans “cpr3” (AAA98789); C. elegans “cpr4” (AAA98785); C. elegans “cpr5” (P43509); C. elegans “CPR6” (AAC70871); L. mexicana (CAA88490); L. major (AAB48119); S. peregrina (S38939); G. gallus (P43233); N. rustica (S60479); B. taurus (AAA80198); A. aegypti (AAA79004); T. cruzi (AAD03404); Human (NP_{—}001899); U. caupo (AAA74445); G. intestinalis. Hemoglobinases: N. americanus (CAB53367); S. japonicum “cathepsin-B like cp”, (S31909): S. japonicum cath B, (S31907); S. japonicum “cathpB”, (CAA50305); S. japonicum, (P43157); S. mansoni “SM31 prec”, (P25792); S. mansoni “cathepsin b”, (AAA29865); A. suum, (AAB40605); A. caninum, (AAC46877); A. caninum, (AAC46878); A. ceylanicum, (AAD17287); H contortus “AC-1”, (AAA29175); H. contortus “AC-2”, (AAA29171); H. contortus “AC-3”, (D48435); H. contortus “AC-4”, (C48435); H. contortus “AC-5”, (B48435); H. contortus “GCP7”, (AAC05262); O. ostertagi “cath B-1 prec”, (P25802); O. ostertagi “cath-B like CP”, (A48454); O. ostertagia “CP-3”, (B48454); O. ostertagia “CB-like”, (AAA29435); O. ostertagia “CathB like”, (AAA29436).

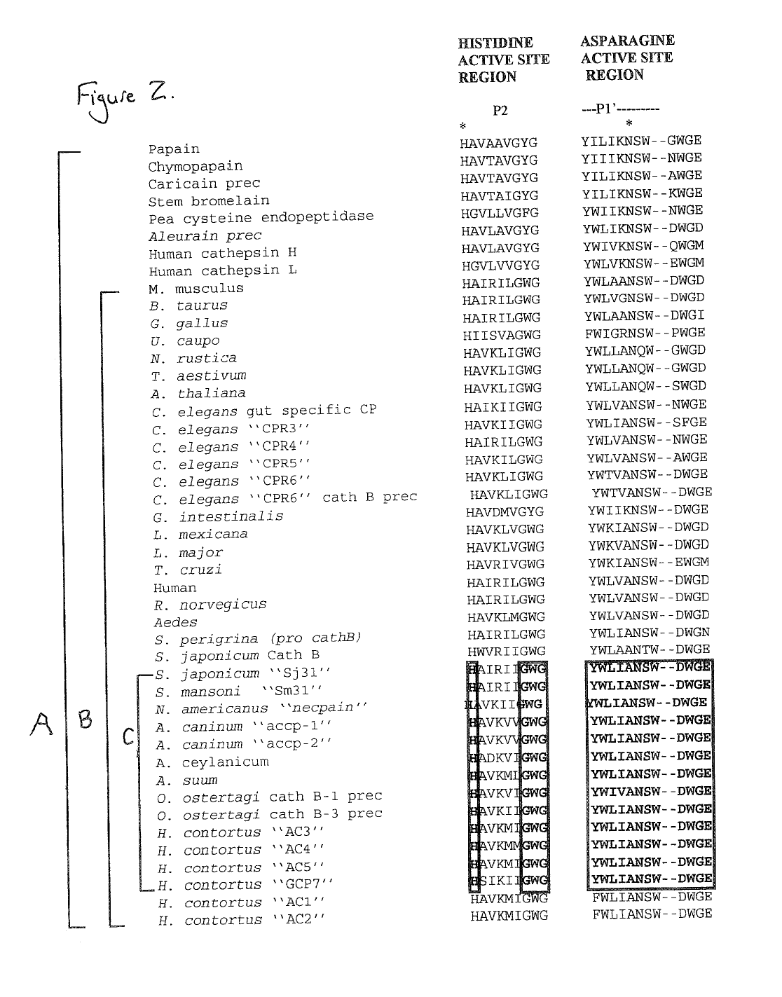

### DETAILED DESCRIPTION OF THE INVENTION

The present invention provides inhibitors that decrease the activity of hemoglobinases, as well as methods of designing or identifying such inhibitors and methods of making them. As used herein, a hemoglobinase is a polypeptide expressed by a parasite, preferably a helminth, that cleaves a peptide bond of one of the polypeptides that make up a hemoglobin molecule, i.e, the α chain or the p chain. “Hemoglobinase activity” refers to the ability of the hemoglobinase to cleave a hemoglobin polypeptide. The term “polypeptide,” as used herein, refers to a polymer of amino acids and does not refer to a specific length of a polymer of amino acids. Thus, for example, the terms peptide, oligopeptide, protein, protease, proteinase, enzyme, and peptidomimetic are included within the definition of polypeptide. This term also includes polypeptides that have been post-translationally modified, for example, by glycosylation, acetylation, phosphorylation and the like. A polypeptide can be isolated from its native source of produced using recombinant techniques, or chemically or enzymatically synthesized.

The term “parasite,” as used herein, includes bloodfeeding endoparasites, such as protozoans and helminths, and bloodfeeding ectoparasites, such as Aedes aegypti and Sarcophaga perigrina. The term “protozoan,” as used herein, refers to a type of parasite that belongs to the phylum Protozoa, including the subphylums Sarcomastigophora, Sporozoa, Ciliophora, and Microspora. The term “helminth,” as used herein, refers to a type of parasite that belongs to the phyla Platyhelminthes (including the classes Turbellaria, Trematoda, Cestoidea, and Monogenea), Nemotoda, Acanthocephala, and Pentastomida. Without intending to be limiting, examples of helminths include, for example, Schistosoma japonicum, S. mansoni, Necater americanus, Ancylostoma caninum, A. ceylanicum, Ascaris suum, Ostertagia ostertagi, Haemenchus contortus, and Fasciola hepatica.

Preferably, the hemoglobinase is produced by S. japonicum, (depicted at GenBank accession No. P43157); S. mansoni, (depicted at GenBank accession No. P25792); A. suum, (depicted at GenBank accession No. AAB40605); A. caninum, (depicted at GenBank accession No. AAC46877); A. caninum, (depicted at GenBank accession No. AAC46878); H. contortus, (depicted at GenBank accession No. D48435); H. contortus, (depicted at GenBank accession No. C48435); H. contortus, (depicted at GenBank accession No. B48435); H. contortus, (depicted at GenBank accession No. AAC05262); O. ostertagi, (depicted at GenBank accession No. P25802).

A host animal is an animal infected with a parasite, or susceptible to infection by a parasite, preferably a helminth. Animals include mammals, lower vertebrates including fish, and birds including domesticated fowl. Preferably, a host animal is a mammal, including, for example, a human, cow, sheep, pig, horse, or goat.

A preferred hemoglobinase is a cathepsin B cysteine protease. The hemoglobinase includes an active site that includes three regions; a cysteine active site region, a histidine active site region, and an asparagine active site region. The term “active site” or “catalytic site,” as used herein, refers to a region of a hemoglobinase, that, as a result of its three dimensional shape, favorably associates with another chemical entity, for instance a substrate hemoglobin or an inhibitor. The term “associates with” refers to a condition of proximity between a chemical entity, or portions thereof, and the hemoglobinase or portions thereof. The association may be non-covalent, wherein the juxtaposition is energetically favored by hydrogen bonding, van der Waals forces, or electrostatic interactions, or it may be covalent. For instance, a portion of a hemoglobin molecule can associate with the active site of a hemoglobinase. The term “chemical entity,” as used herein, refers to chemical compounds, complexes of two or more chemical compounds, and fragments of such compounds or complexes.

In some aspects of the invention, the histidine active site region typically is one of the amino acid sequences depicted in X¹X²H X³ X⁴X²X⁵X⁵GX²X⁶ (SEQ ID NO:1), where X¹ is Leu, Ile, Val, Met, Gly, Ser, Thr, Ala, or Asn, X² is any amino acid, X³ is Gly, Ser, Ala, Cys, or Glu, X⁴ is Leu, Val, Ile, or Met, X⁵ is Leu, Ile, Val, Met, Ala, or Thr, and X⁶ is Gly, Ser, Ala, Asp, Asn, or His. In other aspects of the invention, the histidine active site region typically is one of the amino acid sequences depicted in HX³X⁴X²X⁵X⁷GWG (SEQ ID NO:6), where X³ is either of Ser or Ala, X⁴ is either of Val or Ile, X² is either of Arg or Lys, X⁵ is Ile, Val, or Met, and X⁷ is Ile, Val, Met, or Leu.

In some aspects of the invention, the asparagine active site region is one of the amino acid sequences depicted in by X⁴WX¹X²X³NSWX⁵X⁵X⁵X⁶GX⁵, (SEQ ID NO:2), where X⁴ is Phe or Tyr; X¹ is Leu, Ile, Thr, or Lys; X² is either Ile, Val, Leu, or Ala; X³ is either Ala or Gln; X⁵ is any amino acid; and X⁶ is Phe or Trp. In other aspects of the invention, the asparagine active site region is one of the amino acid sequences depicted in YWIIKNSWX¹X¹DWGE (SEQ ID NO:3), where X¹ is any amino acid; YWX¹X²ANSWX³X³DWGX⁴ (SEQ ID NO:4), where X¹ is either Leu or Ile, x² is Ile or Val, X³ is any amino acid, and X⁴ is Glu, Asn, or Asp; or YWX¹X²ANSWX³X³DWGE (SEQ ID NO:5) where X¹ is either Leu or Ile, x² is lie or Val, and X³ is any amino acid. Most preferably, the asparagine active site region is one of the amino acid sequences depicted in SEQ ID NO:5.

Some aspects of the present invention include analogs and fragments of a hemoglobinase. An “analog” of a hemoglobinase includes at least a portion of the polypeptide, wherein the portion contains deletions or additions of one or more contiguous or noncontiguous amino acids, or containing one or more amino acid substitutions. Substitutes for an amino acid in the polypeptides of the invention are preferably conservative substitutions, which are selected from other members of the class to which the amino acid belongs. For example, it is well-known in the art of protein biochemistry that an amino acid belonging to a grouping of amino acids having a particular size or characteristic (such as charge, hydrophobicity and hydrophilicity) can generally be substituted for another amino acid without substantially altering the structure of a polypeptide. For example, nonpolar (hydrophobic) amino acids include alanine, leucine, isoleucine, valine, proline, phenylalanine, tryptophan, and tyrosine. Polar neutral amino acids include glycine, serine, threonine, cysteine, tyrosine, asparagine and glutamine. Positively charged (basic) amino acids include arginine, lysine and histidine. Negatively charged (acidic) amino acids include aspartic acid and glutamic acid. Examples of preferred conservative substitutions include Lys for Arg and vice versa to maintain a positive charge; Glu for Asp and vice versa to maintain a negative charge; Ser for Thr so that a free —OH is maintained; and Gln for Asn to maintain a free NH².

Hemoglobinase analogs, as that term is used herein, also include modified polypeptides. Modifications of polypeptides of the invention include chemical and/or enzymatic derivatizations at one or more constituent amino acid, including side chain modifications, backbone modifications, and—and C-terminal modifications including acetylation, hydroxylation, methylation, amidation, and the attachment of carbohydrate or lipid moieties, cofactors, and the like.

A “fragment” of a hemoglobinase includes a portion of a hemoglobinase that is at least about 14 amino acids in length. Preferably, the hemoglobinase fragment includes one of the amino acid sequences of SEQ ID NO:2 or SEQ ID NO:6. In other aspects, the hemoglobinase fragment is one of the amino acid sequences of SEQ ID NO:3, SEQ ID NO:4, or SEQ ID NO:5, most preferably SEQ ID NO:5. In some aspects, the hemoglobinase fragment preferably includes one of the amino acid sequences of SEQ ID NO:5 or SEQ ID NO:6.

Methods of Identifying Inhibitors

The invention is further directed to methods for identifying inhibitors of hemoglobinase activity. As used herein, the term “inhibitor” refers to a chemical entity that associates with a hemoglobinase such that the hemoglobinase activity is decreased. An inhibitor can be allosteric, such that it associates with a site that is remote from the active site, or is can be direct, i.e., it associates with the active site of the hemoglobinase. Preferably, an inhibitor is direct.

In one aspect of the invention, an inhibitor of a hemoglobinase can be identified by combining a chemical entity, the hemoglobinase that is to be inhibited, and hemoglobin under conditions suitable for the cleavage of the hemoglobin. There are several methods known to the art for assaying hemoglobinase activity. For instance, hemoglobinase activity can be measured by western immunoblot, as described in Example 2. Other methods that can be used for assaying hemoglobinase activity include those described by Grant et al., (Comp. Biochem. Physiol., 38B, 663-678 (1971)), Chappell et al., (Exp. Parasitol., 61, 160-167 (1986)), and Bogitsh et al., (J. Parasitol., 78, 454-459 (1992)), and mass spectroscopy. After a suitable period of time, the amount of hemoglobin remaining is measured and compared to the amount of hemoglobin remaining when no chemical entity is added. The presence of more hemoglobin in the sample containing both hemoglobinase and chemical entity than in the sample containing hemoglobinase and no chemical entity indicates the chemical entity is an inhibitor. Optionally, after a suitable period of time the amount of hemoglobin remaining is measured and compared to the amount of hemoglobin remaining when no hemoglobinase is added. In increasing order of preference, greater than about 10%, greater than about 50%, greater than about 70%, greater than about 90%, most preferably greater than about 95% of the hemoglobin originally combined with the chemical entity and hemoglobinase is present after the incubation.

Preferably, the inhibitors of the present invention do not inhibit cathepsin B cysteine proteases produced by a host animal to as great a degree as they inhibit a hemoglobinase. Most preferably, the inhibitors of the present invention do not inhibit the cysteine protease human liver cathepsin B (E. C. 3.4.22.1) (Klinkert et al., FEBS Lett., 351, 397-400 (1994)) to as great a degree as they inhibit a hemoglobinase. It is expected that cathepsin B cysteine proteases produced by a host animal are able to cleave hemoglobin, but at a lower rate than a hemoglobinase. Whether an inhibitor of a hemoglobinase also inhibits a cathepsin B cysteine protease produced by a host animal can be measured. Preferably an inhibitor, a cathepsin B cysteine protease produced by a host animal, and a substrate of the cathepsin B cysteine protease are incubated under conditions and for a time period suitable for the cleavage of the substrate. At the end of the time period, the amount of substrate remaining can be measured by western immunoblot, or by mass spectroscopy. Examples of substrates that can be used include the alpha chain of hemoglobin. Preferably, an inhibitor does not inhibit the cathepsin B cysteine protease produced by a host animal. More preferably, greater than 50%, most preferably greater than 90% of the substrate originally combined with inhibitor and cathepsin B cysteine protease, preferably human liver cathepsin B, is present after the incubation.

It is expected that the methods to identify inhibitors are not limited by the type of hemoglobin used. For instance, the hemoglobin can be in the tetramer form, or one of the individual chains, the α chain or β chain, can be used. Preferably, the hemoglobin used is obtained from the animal that is a host for the parasite that produces that hemoglobinase used in the methods to identify an inhibitor. For instance, the host animal of S. mansoni is human. Thus, when the hemoglobinase of S. mansoni is used in the methods to identify an inhibitor, the hemoglobin is preferably obtained from a human.

Hemoglobin can be obtained directly from a host animal using methods known in the art. Alternatively, hemoglobin is obtained from a commercial source, including, for instance, Sigma Chemical Co. (St. Louis, Mo.).

The inhibitors that can be used in the methods described herein include, for example, polypeptides (including, for instance, antibodies), and other non-polypeptide organic compounds or inorganic compounds. Candidate inhibitors can be obtained from various sources. For instance, inhibitors can be naturally produced and obtained from, for instance, microbes, plants, or animals. Complex samples obtained from an extract of a microbe, plant, or animal can be screened for inhibitor activity as described herein. If inhibitor activity is discovered, the inhibitor can optionally be isolated from a complex sample using methods known in the art. An “isolated” inhibitor, such as a polypeptide, non-polypeptide organic compound or inorganic compound, is an inhibitor that has been either removed from its natural environment, produced using recombinant techniques, or chemically or enzymatically synthesized. Preferably, an inhibitor of this invention is purified, i.e., essentially free from any other inhibitors, associated cellular products, or other impurities.

An example of a naturally produced inhibitor is an antibody that binds to an epitope of a hemoglobinase. As used herein, an “epitope” of a hemoglobinase is a portion of a hemoglobinase to which an antibody binds. An epitope can be a series of amino acid residues located adjacent to one another in the primary sequence of the hemoglobinase. Alternatively, an epitope can be made up of amino acid residues that are not located adjacent to one another in the primary sequence of the hemoglobinase, but are positioned together in the three dimensional structure of the hemoglobinase. In some aspects, an epitope to which an antibody binds includes at least one of the amino acids of SEQ ID NO:2 or SEQ ID NO:6. In other aspects, an epitope to which an antibody binds includes SEQ ID NO:3, SEQ ID NO:4, or SEQ ID NO:5, most preferably SEQ ID NO:5. In some aspects of the invention, an epitope to which an antibody binds preferably includes at least one of the amino acids of SEQ ID NO:5 or SEQ ID NO:6.

The antibody can be a polyclonal antibody or a monoclonal antibody. Laboratory methods for producing polyclonal and monoclonal antibodies are known in the art (see, for instance, Harlow E. et al. Antibodies: A laboratory manual Cold Spring Harbor Laboratory Press, Cold Spring Harbor (1988) and Ausubel, R. M., ed. Current Protocols in Molecular Biology (1994)).

A hemoglobinase, including analogs and fragments thereof, can be used to produce monoclonal and polyclonal antibodies using methods known to the art. Antibodies can be screened to determine if they function as an inhibitor of hemoglobinase activity. In some aspects, one of the amino acid sequences of SEQ ID NO:2 or SEQ ID NO:6 is used to produce antibodies. In other aspects, one of the amino acid sequences of SEQ ID NO:3, SEQ ID NO:4, or SEQ ID NO:5, more preferably SEQ ID NO:5, is used to produce antibodies. In some aspects, preferably one of the amino acid sequences of SEQ ID NO:5 or SEQ ID NO:6 is used. More preferably a hemoglobinase that includes one of the amino acid sequences of SEQ ID NO:2 or SEQ ID NO:6 is used. In other aspects, a hemoglobinase that includes one of the amino acid sequences of SEQ ID NO:3, SEQ ID NO:4, or SEQ ID NO:5, most preferably SEQ ID NO:5, is used. In some aspects, preferably a hemoglobinase that includes one of the amino acid sequences of SEQ ID NO:5 or SEQ ID NO:6 is used.

Analogs and fragments that are not themselves antigenic can be coupled to an immunogenic carrier polypeptide to initiate an immune response in the animal or cell. Such non-antigenic fragments, known as haptens, react specifically with an antibody but do not stimulate antibody production unless complexed with a carrier polypeptide. Linking the hapten to a carrier polypeptide produces an immunogen that stimulates antibody production against the hapten. The hapten can be chemically coupled to the carrier polypeptide or a fusion polypeptide can be produced using recombinant genetic methods.

Inhibitors can also be made using recombinant techniques, or chemical or enzymatic synthetic methods. For instance, a polypeptide known to be, or predicted to be, an inhibitor can be produced by a microbe containing a polynucleotide that encodes polypeptide. Chemical or enzymatic synthetic methods known to the art can be used to produce inhibitors. Recombinant techniques or chemical or enzymatic synthetic methods can be used to construct combinatorial libraries of chemical entities that can then be screened for the presence of inhibitors.

In another aspect of the invention, an inhibitor of a hemoglobinase can be identified by rational drug design. For example, the hemoglobinase from S. mansoni, Sm31 (Klinkert et al., FEBS Lett., 351, 397-400 (1994); available at the SWISS-MODEL Repository, www.expasy.ch, SWISS-PROT AC Code P25792_C00001), has been modeled after the x-ray crystal structure of human liver cathepsin B (Musil et al. EMBO J, 10, 2321-2330 (1991); Protein Data Bank Id: 1HUC). X-ray crystal structure coordinates can be used in modeling algorithms known to the art to deduce feasible geometric alignments to produce chemical entities that are sterically and energetically complementary to the hemoglobinase active site. Molecular docking programs which employ approximate potential functions for the deduction of the most functional intermolecular attractions include Shoichet et al., (J. Computational Chem., 13, 380-97 (1992)), and Kuntz et al., (J. Mol. Biol., 161, 269-288 (1982)). Optionally, candidate inhibitors identified in this way from rationale drug design using structure coordinates generated from x-ray diffraction or nuclear magnetic resonance or any other suitable spectroscopic or electromagnetic technique can be synthesized using methods known to the art and tested as described herein.

Methods of Using Inhibitors

The invention is further directed to methods for inhibiting the activity of a hemoglobinase. In one aspect of the present invention, a hemoglobinase can be contacted with an inhibitor, preferably an inhibitor that associates with an amino acid present in a histidine active site region and/or an arginine active site region of the hemoglobinase. Preferably, the active site region includes one of the amino acid sequences of SEQ ID NO:2 or SEQ ID NO:6. In other aspects, the active site is one of the amino acid sequences of SEQ ID NO:3, SEQ ID NO:4, or SEQ ID NO:5, most preferably SEQ ID NO:5. In some aspects, the active site preferably includes one of the amino acid sequences of SEQ ID NO:5 or SEQ ID NO:6. The hemoglobinase can be present in vitro, for instance in an assay as described herein, or in vivo, for instance in an animal. It is expected that decreasing the activity of a hemoglobinase in vivo causes a parasite to be less competitive in the host animal.

Accordingly, another aspect of the present invention provides methods for treating an animal at risk of a parasite infection. Treatment can be prophylactic or, alternatively, can be initiated after infection with a parasite (i.e., therapeutic). Treatment that is prophylactic or therapeutic is referred to herein is treatment of an animal that is at risk of parasite infection. Accordingly, an animal can be treated after it has been diagnosed as being infected with a parasite. Alternatively, an animal that is likely to be exposed to a parasite (e.g., the animal lives in an area where a parasite is endemic) can be treated. Preferably the parasite is a helminth.

The method can include administering to the animal an inhibitor that decreases the activity of a hemoglobinase produced by the parasite. Preferably, the inhibitor is administered with a pharmaceutically acceptable carrier. Pharmaceutically acceptable carriers are described herein.

Another aspect of the invention is directed to immunizing an animal at risk of a parasitic infection. An animal can be immunized by administering antibodies to the animal. This is often referred to in the art as passive immunization. The antibodies can be monoclonal or polyclonal. The antibodies associate with a hemoglobinase, more preferably a hemoglobinase expressed by a helminth. Preferably, the hemoglobinase includes one of the amino acid sequence of SEQ ID NO:2 or SEQ ID NO:6. In other aspects, the hemoglobinase includes one of the amino acid sequence of SEQ ID NO:3, SEQ ID NO:4, or SEQ ID NO:5, most preferably SEQ ID NO:5. In some aspects, the hemoglobinase preferably includes one of the amino acid sequence of SEQ ID NO:5 or SEQ ID NO:6. Preferably, the antibodies associate with at least one amino acid of the amino acid sequences of SEQ ID NO:2 or SEQ ID NO:6. In other aspects, the antibodies associate with at least one amino acid of the amino acid sequences of SEQ ID NO:3, SEQ ID NO:4, or SEQ ID NO:5, most preferably SEQ ID NO:5. In some aspects, the antibodies preferably associate with at least one amino acid of the amino acid sequences of SEQ ID NO:2 or SEQ ID NO:6. Optionally and preferably, the antibodies inhibit hemoglobinase activity.

An animal can also be immunized by administering to an animal at risk of a parasitic infection a hemoglobinase, analog or fragment thereof. Preferably, the animal is immunized with a fragment having one of the amino acid sequences of SEQ ID NO:2 or SEQ ID NO:6. In other aspects, the animal is immunized with a fragment having one of the amino acid sequences of SEQ ID NO:3, SEQ ID NO:4, or SEQ ID NO:5, most preferably SEQ ID NO:5. In some aspects, the animal is immunized with a fragment having one of the amino acid sequences of SEQ ID NO:5 or SEQ ID NO:6. More preferably the animal is immunized with a hemoglobinase that includes one of the amino acid sequences of SEQ ID NO:2 or SEQ ID NO:6. In other aspects, the animal is immunized with a hemoglobinase that includes one of the amino acid sequences of SEQ ID NO:3, SEQ ID NO:4, or SEQ ID NO:5, most preferably SEQ ID NO:5. In some aspects, the animal is immunized with a hemoglobinase that includes one of the amino acid sequences of SEQ ID NO:5 or SEQ ID NO:6. Preferably, the hemoglobinase, analog or fragment thereof is administered with an adjuvant to non-specifically stimulate an immune response. Adjuvants are known to the art and include, for instance, Freund's incomplete adjuvant and Freund's complete adjuvant.

The present invention further provides a pharmaceutical composition that includes, for instance, an inhibitor and a pharmaceutically acceptable carrier. The compositions of the present invention are formulated in pharmaceutical preparations in a variety of forms adapted to the chosen route of administration. Formulations include those suitable for oral administration or parental administration, including, for example, subcutaneous, intramuscular, and intravenous.

The formulations may be conveniently presented in unit dosage form and may be prepared by methods well known in the art of pharmacy. All methods of preparing a pharmaceutical composition include the step of bringing the active compound (e.g., an inhibitor) into association with a carrier which constitutes one or more accessory ingredients. In general, the formulations are prepared by uniformly and intimately bringing the active compound into association with a liquid carrier, a finely divided solid carrier, or both, and then, if necessary, shaping the product into the desired formulations.

Formulations suitable for parenteral administration conveniently comprise a sterile aqueous preparation of the composition, or dispersions of sterile powders that include the composition, which are preferably isotonic with the blood of the recipient. Isotonic agents that can be included in the liquid preparation include sugars, buffers, and sodium chloride. Solutions of the composition can be prepared in water, optionally mixed with a nontoxic surfactant. Dispersions of the composition can be prepared in water, ethanol, a polyol (such as glycerol, propylene glycol, liquid polyethylene glycols, and the like), vegetable oils, glycerol esters, and mixtures thereof The ultimate dosage form is sterile, fluid and stable under the conditions of manufacture and storage. The necessary fluidity can be achieved, for example, by using liposomes, by employing the appropriate particle size in the case of dispersions, or by using surfactants. Sterilization of a liquid preparation can be achieved by any convenient method that preserves the bioactivity of the composition, preferably by filter sterilization. Preferred methods for preparing powders include vacuum drying and freeze drying of the sterile injectable solutions. Subsequent microbial contamination can be prevented using various antimicrobial agents, for example, antibacterial, antiviral and antifungal agents including parabens, chlorobutanol, phenol, sorbic acid, thimerosal, and the like. Absorption of the composition by the animal over a prolonged period can be achieved by including agents for delaying, for example, aluminum monostearate and gelatin.

In addition to the aforementioned ingredients, the formulations of this invention may further include one or more accessory ingredients including diluents, buffers, binders, disintegrants, surface active agents, thickeners, lubricants, preservatives (including antioxidants) and the like.

The present invention is illustrated by the following examples. It is to be understood that the particular examples, materials, amounts, and procedures are to be interpreted broadly in accordance with the scope and spirit of the invention as set forth herein.

### EXAMPLE 1

### Identification of the Hemoglobinase Motif

A stepwise analysis was performed to identify a motif present in hemoglobinases of bloodfeeder helminths. To begin, each cysteine protease known to exist in parasites was evaluated for the hypothesized roles of the cysteine proteases in infection by the parasite. Hypothesized roles included IgG degradation, interleukin-2 degradation, processing of precursor protein for increased pathogenicity, and hemoglobin degradation.

Next, the existence of specific phenotypic characteristics in this population of cysteine proteases was determined. Three phenotypic characteristics were used: IgG degradation by parasites, pH dependence of certain cysteine proteases, and hemoglobin degradation by parasites. Cysteine proteases of Schistosoma japonicum, S. mansoni, Ostertagia ostertagi, and Haemenchus contortus were found to be characterized as hemoglobinases.

Cysteine proteases in this group were divided based on which cysteine protease class to which each cysteine protease belonged, i.e., cathepsin L, cathepsin H, and cathepsin B. The next step was to compare the amino acid sequence of the cysteine proteases in each of these classes. Thus, only those cysteine proteases with a known primary amino acid sequence were compared. Since there were not enough primary sequences available for cathepsin L or cathepsin H cysteine proteases that were thought to be involved in hemoglobin degradation, only the primary amino acid sequences of cathepsin B proteases thought to be involved in hemoglobin degradation were compared.

Cathepsin B enzymes are generally known to have general housekeeping functions, i.e., they are typically located in lysosomes and involved in normal cellular degradation pathways. It was hypothesized that the fine tuning of a housekeeping enzyme like cathepsin B for a specialized function such as hemoglobin degradation would most likely occur in the active site region where subtle changes can cause modifications in substrate specificity. Accordingly, only the regions of the active site were analyzed. Each of the three active site regions known to exist in cysteine proteases, i.e., the cysteine active site region, the histidine active region, and the asparagine active site region, were analyzed.

The amino acid sequences of the active sites were aligned using the Multiple Alignment Construction and Analysis Workbench (MACAW), version 2.0.4 Segment pair overlap search for blocks was used with the pairwise score cutoff of 44, and the minimum sequences per block at 2. Unlike conventional alignment programs like Clustal, this alignment utility makes separate alignments in different regional subsets. The resulting alignments were further refined by looking at each computer-generated alignment and manually modifying it to maximize the conserved features exclusively located in the active site regions. This analysis resulted in the identification of a motif (depicted at SEQ ID NO:5) present in the asparagine active site region.

### EXAMPLE 2

### Measurement of Hemoglobinase Activity

If a drug is to be designed against the hemoglobinase motif, there are several experimental methods which can be utilized to test it. Several of those methods will now follow:

The amount of hemoglobin present in a sample can be determined by Western blot. Hemoglobin (human, goat or sheep, as these are hosts of the bloodfeeding helminths) was obtained from Sigma (St. Louis, Mo.). Hemoglobinase was obtained by the method of Grant et al., (Comp. Biochem. Physiol., 38B, 663-678 (1971)), Chappell et al., (Exp. Parasitol., 61, 160-167 (1986)), or Bogitsh et al., (J. Parasitol., 78, 454-459 (1992)). About 1 μg of hemoglobin and about 5 μg of hemoglobinase was incubated in 0.2M citrate buffer (pH 5.5) supplemented with 10 mM cysteine. Incubation was for about 18 hours at 37° C., under sterile conditions. A control tube included hemoglobin without added hemoglobinase. After incubation, the contents of the tubes were resolved on a 12.5% sodium dodecyl sulfate-polyacrylamide gel (SDS-PAGE) under nonreducing conditions (i.e., no 2-mercaptoethanol is added, and the mix is not boiled). The use of nonreducing conditions maintains the integrity of the 66 kDa hemoglobin tetramer. After the gel was been finished, it was transferred to PVDF membrane (Amersham, Piscataway, N.J.) by following the manufacturer's suggested procedure.

The blot was probed for one hour using a biotin labeled primary antibody (goat anti-human α-chain hemoglobin, Sigma) in 0.03% bovine serum albumin with 0.05% Tween-20 in 0.1 M PBS, pH 7.2. The antibody was diluted 1:10000 in the buffer. Incubation was for 1 hour at room temperature. The blot was than washed with the 0.05% Tween-20/0.1 M PBS, pH 7.2 solution for one hour. A secondary antibody (goat anti-goat IgG, Sigma) conjugated to strepavidin and peroxidase was incubated with the blot for one hour. The secondary antibody is dissolved in 0.05% Tween-20/0.1 M PBS, pH 7.2. The blot was washed for one hour in 0.05% Tween20/0.1 M PBS, pH 7.2, followed by visualization using an enhanced chemiluminescence kit (Amersham) following the manufacturer's suggested procedure. Hemoglobinase activity was identified by the decrease in the amount of the 66 kDa tetramer band by degradation compared to the amount of hemoglobin in the control tubes without hemoglobinase added.

The complete disclosure of all patents, patent applications, and publications, and electronically available material (e.g., GenBank amino acid and nucleotide sequence submissions) cited herein are incorporated by reference. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

What is claimed is:

1. A method for treating an animal at risk of a parasite infection comprising administering to the animal an inhibitor that decreases the activity of a parasite hemoglobinase.

2. The method of claim 1 wherein the hemoglobinase comprises an asparagine active site region.

3. The method of claim 2 wherein the asparagine active site region comprises an amino acid sequence selected from the group consisting of amino acid sequences of SEQ ID NO:5.

4. The method of claim 1 wherein the inhibitor associates with an amino acid of an amino acid sequence selected from the group consisting of amino acid sequences of SEQ ID NO:5 and SEQ ID NO:6.

5. The method of claim 1 wherein the inhibitor is a chemical entity selected from the group consisting of a polypeptide, a non-polypeptide organic compound, and an inorganic compound.

6. The method of claim 5 wherein the polypeptide is selected from the group consisting of a polyclonal antibody and a monoclonal antibody.

7. The method of claim 1 wherein the animal is a mammal.

8. The method of claim 1 wherein the parasite is a helminth.

9. A method for treating an animal at risk of a helminth infection comprising administering to the animal an inhibitor that decreases the activity of a helminth hemoglobinase.

10. A method for treating an animal at risk of a parasite infection comprising administering to the animal an inhibitor that decreases the activity of a hemoglobinase comprising an asparagine active site region having an amino acid sequence selected from the group consisting of SEQ ID NO:3, SEQ ID NO:4, and SEQ ID NO:5, wherein the inhibitor associates with an amino acid of the asparagine active site region.

11. A method for treating an animal at risk of a parasite infection comprising administering to the animal an inhibitor that decreases the activity of a hemoglobinase comprising an asparagine active site region having an amino acid sequence SEQ ID NO:5, wherein the inhibitor associates with an amino acid of the asparagine active site region.

12. A method for treating an animal at risk of a parasite infection comprising administering to the animal an inhibitor that decreases the activity of a hemoglobinase comprising a histidine active site region having an amino acid sequence SEQ ID NO:6, wherein the inhibitor associates with an amino acid of the histidine active site region.

13. A method for inhibiting the activity of a parasite hemoglobinase comprising contacting a parasite hemoglobinase with an inhibitor of hemoglobinase activity.

14. The method of claim 13 wherein the hemoglobinase is in vitro.

15. The method of claim 13 wherein the hemoglobinase is present in an animal.

16. A method for inhibiting the activity of a helminth hemoglobinase comprising contacting a helminth hemoglobinase with an inhibitor of hemoglobinase activity.

17. A method for inhibiting the activity of a hemoglobinase comprising contacting a hemoglobinase comprising an asparagine active site region with an inhibitor of hemoglobinase activity, wherein the asparagine active site region has an amino acid sequence selected from the group consisting of SEQ ID NO:3, SEQ ID NO:4, and SEQ ID NO:5, and wherein the inhibitor associates with an amino acid of the asparagine active site region.

18. A method for inhibiting the activity of a hemoglobinase comprising contacting a hemoglobinase comprising an asparagine active site region with an inhibitor of hemoglobinase activity, wherein the asparagine active site region has an amino acid sequence SEQ ID NO:5, and wherein the inhibitor associates with an amino acid of the asparagine active site region.

19. A method for inhibiting the activity of a hemoglobinase comprising contacting a hemoglobinase comprising a histidine active site region with an inhibitor of hemoglobinase activity, wherein the asparagine active site region has an amino acid sequence SEQ ID NO:6, and wherein the inhibitor associates with an amino acid of the histidine active site region.

20. A method for identifying an inhibitor of hemoglobinase activity comprising:
incubating a solution comprising a chemical entity, a hemoglobinase, and hemoglobin under conditions and for a time period suitable for the catabolism of the hemoglobin; and
measuring the amount of hemoglobin remaining at the end of the time period, wherein the presence of more hemoglobin in the solution compared to a comparably treated solution that does not contain the chemical entity indicates the chemical entity is an inhibitor of hemoglobinase activity.

21. The method of claim 20 wherein the hemoglobinase comprises an asparagine active site region.

22. The method of claim 21 wherein the asparagine active site region comprises an amino acid sequence of SEQ ID NO:5.

23. The method of claim 20 wherein the chemical entity is selected from the group consisting of a polypeptide, a peptidomimetic, an organic compound, and an inorganic compound.

24. The method of claim 23 wherein the polypeptide is selected from the group consisting of a polyclonal antibody and a monoclonal antibody.

25. The method of claim 20 wherein the chemical entity associates with an amino acid of an amino acid sequence selected from the group consisting of SEQ ID NO:5 and SEQ ID NO:6.

26. The method of claim 20 wherein the hemoglobinase activity is derived from a parasite and wherein the solution is a first solution, the method further comprising:
incubating a second solution comprising the inhibitor, a cathepsin B cysteine proteinase produced by a host animal, and a substrate of the cathepsin B cysteine proteinase under conditions and for a time period suitable for cleavage of the substrate; and
measuring the amount of substrate remaining in the solution at the end of the time period is measured, wherein the presence of greater than 50% of the substrate at the end of the time period indicates that the inhibitor is a selective inhibitor of the parasite hemoglobinase activity.

27. The method of claim 26 wherein the cathepsin B cysteine proteinase produced by a host animal is human liver cathepsin B.

28. The method of claim 26 wherein the cathepsin B cysteine proteinase substrate is an a chain of hemoglobin.

29. A method for identifying an inhibitor of parasite hemoglobinase activity comprising:
incubating a solution comprising a chemical entity, a parasite hemoglobinase, and hemoglobin under conditions and for a time period suitable for the catabolism of the hemoglobin; and
measuring the amount of hemoglobin remaining at the end of the time period, wherein the presence of more hemoglobin in the solution compared to a comparably treated solution that does not contain the chemical entity indicates the chemical entity is an inhibitor of parasite hemoglobinase activity.

30. A method for identifying an inhibitor of helminth hemoglobinase activity comprising:
incubating a solution comprising a chemical entity, a helminth hemoglobinase, and hemoglobin under conditions and for a time period suitable for the catabolism of the hemoglobin; and
measuring the amount of hemoglobin remaining at the end of the time period, wherein the presence of more hemoglobin in the solution compared to a comparably treated solution that does not contain the chemical entity indicates the chemical entity is an inhibitor of helminth hemoglobinase activity.

31. A method for identifying an inhibitor of hemoglobinase activity comprising:
incubating a solution comprising a chemical entity, a hemoglobinase comprising an asparagine active site region having an amino acid sequence selected from the group consisting of SEQ ID NO:3, SEQ ID NO:4, and SEQ ID NO:5, and hemoglobin under conditions and for a time period suitable for the catabolism of the hemoglobin; and
measuring the amount of hemoglobin remaining at the end of the time period, wherein the presence of more hemoglobin in the solution compared to a comparably treated solution that does not contain the chemical entity indicates the chemical entity is an inhibitor of hemoglobinase activity.

32. A method for identifying an inhibitor of hemoglobinase activity comprising:
incubating a solution comprising a chemical entity, a hemoglobinase comprising an asparagine active site region having an amino acid sequence SEQ ID NO:5, and hemoglobin under conditions and for a time period suitable for the catabolism of the hemoglobin; and
measuring the amount of hemoglobin remaining at the end of the time period, wherein the presence of more hemoglobin in the solution compared to a comparably treated solution that does not contain the chemical entity indicates the chemical entity is an inhibitor of hemoglobinase activity.

33. A method for identifying an inhibitor of hemoglobinase activity comprising:
incubating a solution comprising a chemical entity, a hemoglobinase comprising an histidine active site region having an amino acid sequence SEQ ID NO:6, and hemoglobin under conditions and for a time period suitable for the catabolism of the hemoglobin; and
measuring the amount of hemoglobin remaining at the end of the time period, wherein the presence of more hemoglobin in the solution compared to a comparably treated solution that does not contain the chemical entity indicates the chemical entity is an inhibitor of hemoglobinase activity.

34. A method of immunizing an animal at risk of a parasitic infection comprising administering to the animal an antibody that associates with a region of a hemoglobinase.

35. The method of claim 34 wherein the region of the hemoglobinase comprises an amino acid sequence selected from the group consisting of amino acid sequences of SEQ ID NO:5 and SEQ ID NO:6.

36. The method of claim 34 wherein the antibody is selected from the group consisting of a polyclonal antibody and a monoclonal antibody.

37. The method of claim 34 wherein the parasite is a helminth.

38. A method of immunizing an animal at risk of a helminth infection comprising administering to the animal an antibody that associates with a region of a helminth hemoglobinase.

39. A method for immunizing an animal at risk of a parasitic infection comprising administering to the animal a polypeptide that induces an immune response against a hemoglobinase expressed by a parasite.

40. The method of claim 39 wherein the polypeptide comprises an amino acid sequence selected from the group consisting of amino acid sequences of SEQ ID NO:5 and SEQ ID NO:6.

41. The method of claim 39 wherein the polypeptide has an amino acid sequence selected from the group consisting of amino acid sequences of SEQ ID NO:5 and SEQ ID NO:6.

42. The method of claim 39 wherein the parasite is a helminth.

43. A method for immunizing an animal at risk of a helminth infection comprising administering to the animal a polypeptide that induces an immune response against a hemoglobinase expressed by a helminth.

44. A composition for inducing an immune response in an animal comprising a polypeptide comprising an amino acid sequence selected from the group consisting of amino acid sequences of SEQ ID NO:5 and SEQ ID NO:6.

45. A composition for inducing an immune response in an animal comprising a polypeptide having an amino acid sequence selected from the group consisting of amino acid sequences of SEQ ID NO:5 and SEQ ID NO:6.

46. A computer-assisted method for identifying an inhibitor of parasite hemoglobinase activity comprising:
supplying a computer modeling application with a set of structure coordinates of all or a portion of a parasite hemoglobinase;
supplying the computer modeling application with a set of structure coordinates of a chemical entity; and
determining whether the chemical entity is an inhibitor expected to bind to or interfere with the hemoglobinase, wherein binding to or interfering with the hemoglobinase is indicative of potential inhibition of the hemoglobinase.

47. A computer-assisted method for identifying an inhibitor of helminth hemoglobinase activity comprising:
supplying a computer modeling application with a set of structure coordinates of all or a portion of a helminth hemoglobinase;
supplying the computer modeling application with a set of structure coordinates of a chemical entity; and
determining whether the chemical entity is an inhibitor expected to bind to or interfere with the hemoglobinase, wherein binding to or interfering with the hemoglobinase is indicative of potential inhibition of the hemoglobinase.

48. A computer-assisted method for designing an inhibitor of parasite hemoglobinase activity comprising:
supplying a computer modeling application with a set of structure coordinates of all or a portion of a parasite hemoglobinase;
supplying the computer modeling application with a set of structure coordinates of a chemical entity;
structurally modifying the chemical entity to yield a set of structure coordinates for a modified chemical entity; and
determining whether the modified chemical entity is an inhibitor expected to bind to or interfere with the hemoglobinase, wherein binding to or interfering with the hemoglobinase is indicative of potential inhibition of the hemoglobinase.

49. A computer-assisted method for designing an inhibitor of helminth hemoglobinase activity comprising:
supplying a computer modeling application with a set of structure coordinates of all or a portion of a helminth hemoglobinase;
supplying the computer modeling application with a set of structure coordinates of a chemical entity;
structurally modifying the chemical entity to yield a set of structure coordinates for a modified chemical entity; and
determining whether the modified chemical entity is an inhibitor expected to bind to or interfere with the hemoglobinase, wherein binding to or interfering with the hemoglobinase is indicative of potential inhibition of the hemoglobinase.

50. A computer-assisted method for designing an inhibitor of parasite hemoglobinase activity comprising:
supplying a computer modeling application with a set of structure coordinates of all or a portion of a parasite hemoglobinase;
computationally building a chemical entity represented by a set of structure coordinates; and
determining whether the chemical entity is an inhibitor expected to bind to or interfere with the hemoglobinase, wherein binding to or interfering with the hemoglobinase is indicative of potential inhibition of the hemoglobinase.

51. A computer-assisted method for designing an inhibitor of helminth hemoglobinase activity comprising:
supplying a computer modeling application with a set of structure coordinates of all or a portion of a helminth hemoglobinase;
computationally building a chemical entity represented by a set of structure coordinates; and
determining whether the chemical entity is an inhibitor expected to bind to or interfere with the hemoglobinase, wherein binding to or interfering with the hemoglobinase is indicative of potential inhibition of the hemoglobinase.
